(19) United States
(12) Patent Application Publication
(10) Pub. No.: US 2016/0252487 A1
(43) Pub. Date: Sep. 1, 2016

(54) SENSOR HUB METHOD AND APPARATUS FOR AN ELECTRICAL OUTLET

(71) Applicant: Intel Corporation, Santa Clara, CA (US)

(72) Inventor: Sandhiprakash J. Bhide, Beaverton, OR (US)

(21) Appl. No.: 14/962,662

(22) Filed: Dec. 8, 2015

Related U.S. Application Data
(60) Provisional application No. 62/121,095, filed on Feb. 26, 2015.

(51) Int. Cl. G01N 33/00; G01K 1/14

(52) U.S. Cl. CPC G01N 33/0036; G01K 1/14; G01N 33/0031

(57) ABSTRACT

Apparatuses, methods and storage medium associated with a intelligent sensor hub are disclosed herein. In embodiments, an intelligent sensor hub may include a plurality of sensors, one or more communication interfaces, and a processor coupled to the sensors and the one or more communication interfaces to receive sensor data from the sensors and transmit the sensor data or results of analysis of the sensor data to an external recipient. The senor hub may further include a body encasing the sensors, the one or more communication interfaces and the processor, wherein the body includes features to facilitate the sensor hub to be disposed at an electrical outlet. Other embodiments may be disclosed or claimed.

### RELATED APPLICATION

This application is a non-provisional application of U.S. Provisional Application 62/121,095, entitled “Intelligent Sensor Hub Method and Apparatus,” filed on Feb. 26, 2015, and claims priority to the 62/121,095 provisional application, which specification is hereby fully incorporated by reference.

### TECHNICAL FIELD

The present disclosure relates to the field of sensing technology, in particular, to apparatuses, methods and storage medium associated with a sensor hub.

### BACKGROUND

The background description provided herein is for the purpose of generally presenting the context of the disclosure. Unless otherwise indicated herein, the materials described in this section are not prior art to the claims in this application and are not admitted to be prior art by inclusion in this section.

In today's homes, typically, there are at most one smoke alarms on each floor. A room that has created smoke could be farther away from the smoke detector so the problem is not determined where the smoke occurs. Moreover, people may forget to change the batteries in the smoke detectors. Also, there is normally one temperature sensor for the entire house and as the heat rises higher as a result, sometimes the top floors are hotter than the bottom floor creating a discomfort. Additionally, homes are generally wired for temperature control and not for humidity control, which is also necessary for room comfort.

Further, it is common problem in many homes that furnaces create a Carbon Monoxide (CO) which is an odorless and deadly gas. It is about 3% lighter but diffuses evenly through the room. This gas needs to be detected in every room and in multiple places around the house. In larger rooms, sometimes the vents are located in such a place that one side of the room is colder and the other side is hotter and there is no uniform and comfortable temperature within the room which creates discomfort. And yet, most homes have only one CO sensor.

Still further, Liquefied petroleum gas (LPG) or propane or butane, are flammable mixtures of hydrocarbon gases used as a cooking gas can sometime leak or the control can be left off the OFF position so the LPG leaks. LPG is a highly flammable and it can go undetected. Home may or may not have any LPG sensors.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates a front view of the intelligent sensor hub, here after just called sensor hub, of the present disclosure, in accordance with various embodiments.

FIG. 2 illustrates a side view of the sensor hub of the present disclosure, in accordance with various embodiments.

FIG. 3 illustrates mating of the sensor hub of the present disclosure with a conventional electrical outlet, in accordance with various embodiments.

FIG. 4 illustrates the sensor hub of the present disclosure, in accordance with various alternate embodiments.

FIG. 5 illustrates an example deployment of the sensor hub of the present disclosure, in accordance with various embodiments.

FIG. 6 illustrates a block diagram of an example architecture of the sensor hub of the present disclosure, in accordance with various embodiments.

FIG. 7 illustrates an example computer-readable storage medium with instructions configured to enable a sensor hub to practice aspects of the present disclosure, in accordance with various embodiments.

### DETAILED DESCRIPTION

Apparatuses, methods and storage medium associated with a sensor hub are disclosed herein. In embodiments, a sensor hub may include a plurality of sensors of various sensor types, one or more communication interfaces, and a processor coupled to the sensors and the one or more communication interfaces to receive and process sensor data from the sensors, and to transmit the sensor data or results of analysis of the sensor data to an external recipient. The sensor hub may further include a body encasing the sensors, the one or more communication interfaces and the processor, wherein the body may include features to facilitate the sensor hub to be conveniently disposed at an electrical outlet (from which the sensor hub may draw power).

In embodiments, the sensors may include smoke, CO, LPG, motion, temperature, humidity, smoke and other sensors. The one or more communication interfaces may include one or more low power, small overhead protocols communication interfaces, such as Bluetooth Low Energy (BLE), Z-Wave, or ZigBee.

In embodiments, the body may comprise a casing with a plurality of prongs to facilitate the sensor hub to be mated with a conventional electrical outlet. A small transformer may be included to convert 110/120V (or 240 V) to 3.3V or 5V DC or both depending on the processor and sensors. The body may take the form of a contained button size enclosure (˜1″ in diameter), i.e. the size of an old U.S. dollar coin. In other embodiments, the body may take the form of a face plate of a conventional electrical outlet (with the processor and communication mechanism disposed behind the face plate, and the sensors exposed at the front surface of the face plate). Some face plate embodiments may include wiring that contacts the power lines of the electrical power to derive power. Other face plate embodiments may obtain power through induction thereby eliminating a direct contact with the power line. In either set of implementations, there may always be a rechargeable cell battery that allows sensing even when the electricity is off for a short duration.

The uniqueness of this sensor hub is that it fits right into a conventional electrical receptacle. It is small and houses sensors of interest for a given environment, such as residential housings. However, the sensor hub is applicable to any other environment, such as commercial buildings, office spaces, manufacturing floors, retail spaces, industrial and Smart City and other similar environments. In embodiments, the sensor hub may work in conjunction with external controllers, including proximately located controllers. In embodiments, these proximately located controllers can act as sensor processing unit where sensor analytics can run. One such external controllers is the centrally located intelligent LED bulb which can be the aggregator point in the room or home/office. Intelligent light bulb is the subject matter of co-pending U.S. non-provisional application <attorney docket 127075-205200>, contemporaneously filed. The alternatives may include, but are not limited to desktop computers, laptop computers, computer tablets, servers, smartphones, set-top boxes, game consoles, or intelligent Internet-of-Things (IoT), each one with an adequate capability to do sensor processing.

Homes may be single story or double story dwellings. It is often sensed that upstairs tend to get hotter because the warm air is thinner than cold air and this rises to the top resulting in downstairs which is colder than the upstairs rooms, thereby creating many uncomfortable spots in the home. Depending on where the supply vents, the temperature in area of the same room may be different than another area resulting people shivering on side while feeling hot on the other. Most houses have one or two at most thermostats, thereby using one or two thermostats as the proxy(ies) for the entire house. This results in overhearing in one area and not enough heat in another area. This results in loss of energy efficiency.

Secondly, most houses also have one or two smoke sensors, and at best one Carbon Monoxide sensor, most likely no Liquefied Petroleum Gas sensors. When smoke is created in a room that is farthest from the smoke alarm, it may take several minutes to the smoke to reach the sensors thereby creating delay in emergency response.

In embodiments, a range of 2-3 units per every room or 10-20 of these units can be placed in each home at all kinds of places creating detailed map of the location of these devices and the associated sensor readings. For HVAC systems of the home, it creates not just one or two temperature, and humidity sensors but many sensing points thus creating a much better representation of the home temperature, humidity, and pressure measurement thereby providing a basis for creating uniform comfortable air mass in the rooms. The small sensor logic or a small controller may be run at some prescribed duration so as to be good enough to give information but not so much that it uses too much energy.

The CO and LPG gas sensors in each room provide safety in each room and which dangers to be detected immediately. The sensor hub may be provided with holes on this disk through which the unit is able to breathe different gases. When the house is locked and no one is supposed to at home, the motion sensor may do the job of monitoring the entry into any room when it is not expected.

In the description to follow, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Operations of various methods may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiments. Various additional operations may be performed and/or described operations may be omitted, split or combined in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

As used hereinafter, including the claims, the term “module” may refer to, be part of, or include an Application Specific Integrated Circuit (ASIC), an electronic circuit, a processor (shared, dedicated, or group) and/or memory (shared, dedicated, or group) that execute one or more software or firmware programs, a combinational logic circuit, and/or other suitable components that provide the described functionality.

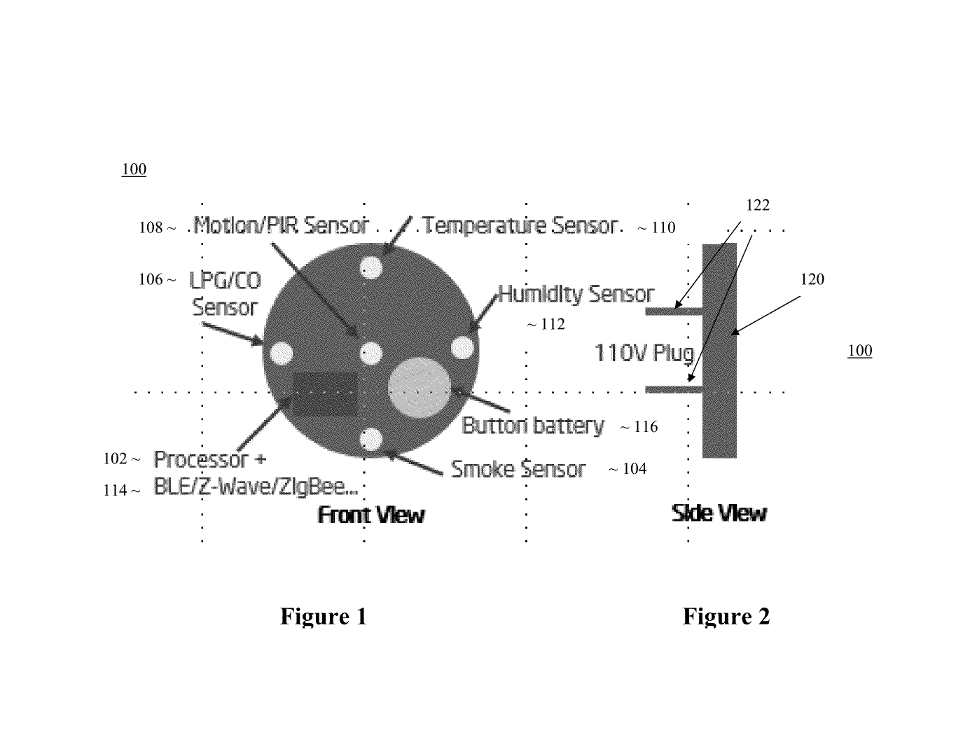

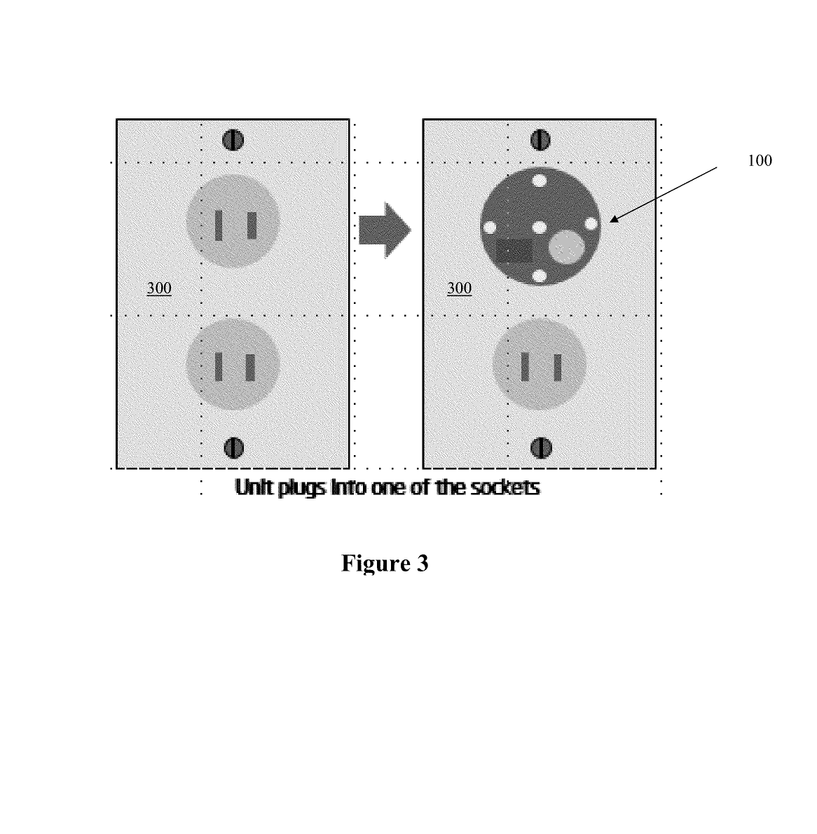

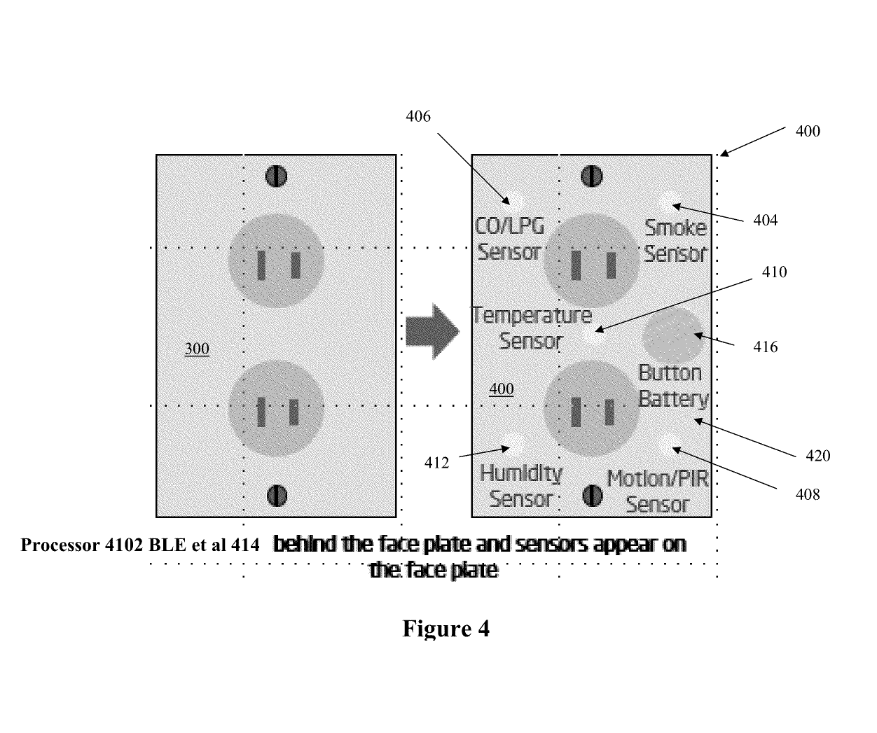

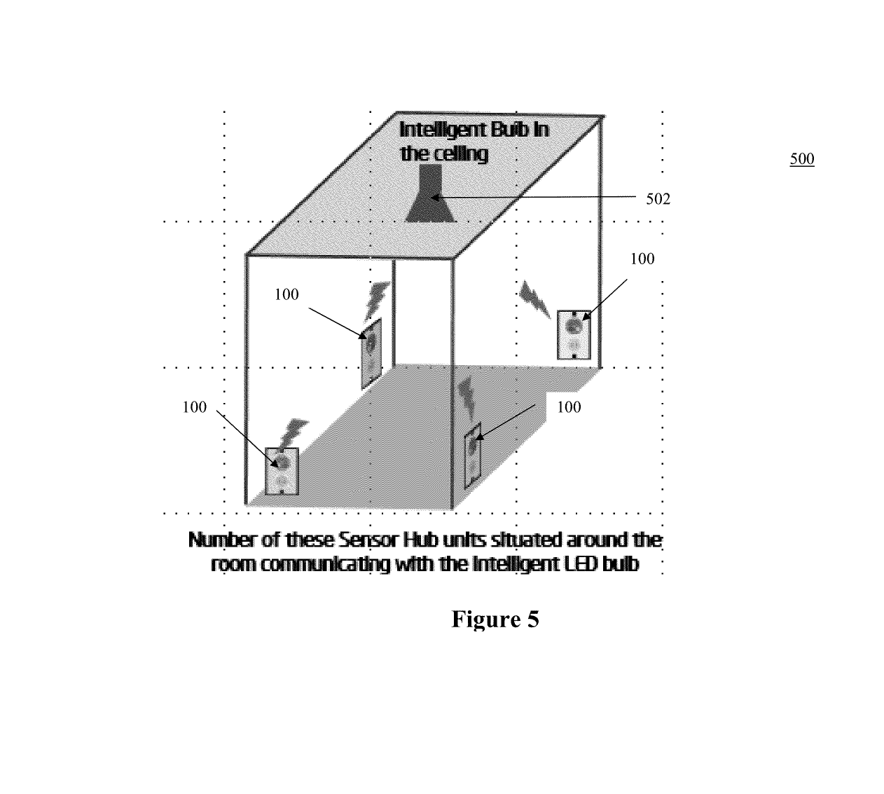

Referring now to FIGS. 1-3 and 5, wherein the sensor hub of the present disclosure, in accordance with various embodiments, are illustrated. More specifically, FIGS. 1-2 illustrates a front view and a side view of the sensor hub of the present disclosure, in accordance with various embodiments. FIG. 3 illustrates the sensor hub mated with a conventional electrical outlet. FIG. 4 illustrates an alternate face plate embodiments of the sensor hub, according to other embodiments. FIG. 5 illustrates an example deployment of a plurality of the sensor hubs of the present disclosure.

As shown in FIG. 1, in embodiment, sensor hub end node (or simply, sensor node) 100 may include a processor 102, various sensors 104-112 of various types (such as, smoke, CO, LPG or cooking gas, motion/passive temperature, humidity, and so forth), and short distance communication mechanism 114, such as Bluetooth Low Energy (BLE), Z-Wave, or ZigBee, or any other communication method and rechargeable battery 116, which is charged either directly from a AC-DC power supply, through induction, using a super capacitor, or a replaceable battery. Various sensors 104-112 (such as, smoke, CO, LPG or cooking gas, motion/passive temperature, humidity, and so forth) may be employed to sense and collect a wide range of environmental data. These elements (processor, sensors, and so forth) 102-116, as shown in FIG. 2, may be enclosed with a body 120 having a number of prongs 122 configured to enable sensor hub 100 to mate with a conventional electrical outlet, and draw power from the electrical outlet. FIG. 3 depicts sensor hub 100 as mated with a conventional electrical outlet 300. In embodiments, a small transformer (not shown) may be provided to convert 110/120V (or 240 v) to 3.3V or 5V DC or both depending on the processor, communication interfaces and sensors' needs. In embodiments, the body 120 may take the form of a contained button size enclosure (˜1″ in diameter), i.e. the size of an old U.S. dollar coin.

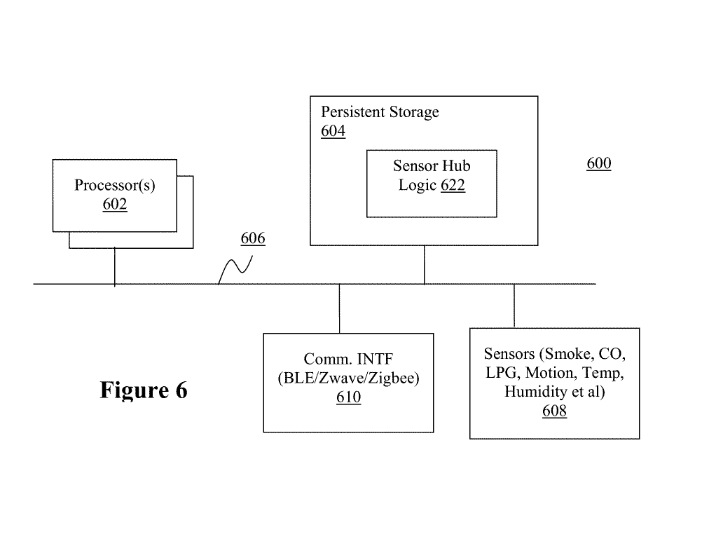

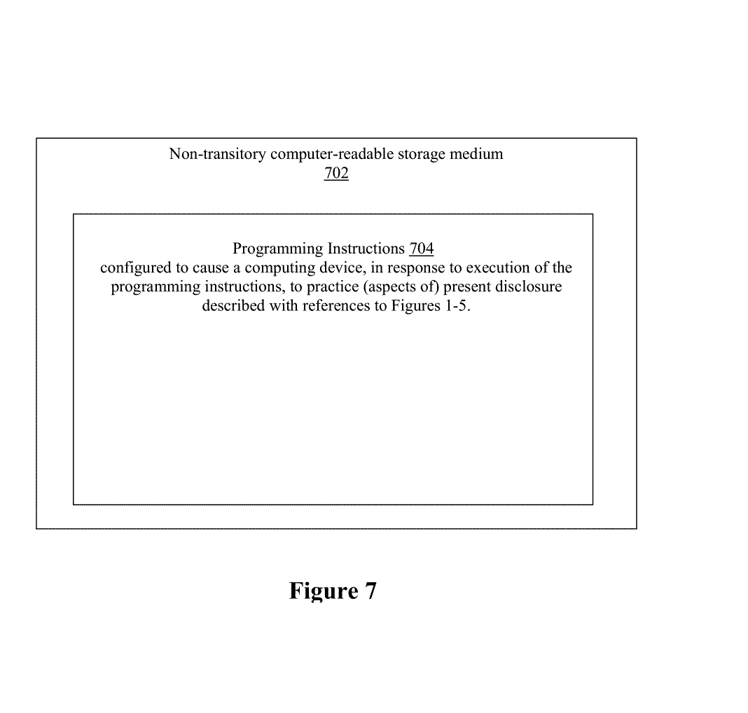

Further, sensor hub 100 may be provided with a small real time executive or a kernel (such as sensor hub logic 622 of FIG. 6). Sensor hub logic 622, in embodiments, may be configured to go around the sensors 104-112 in a round robin fashion, read the sensor data, compare the data with upper and lower control limits or using some algorithm, make a judgment and/or pass this data to an external controller, such as a proximately located controller, as an input for higher level analytics calculation. The higher level analytics calculation may be calculated for HVAC control of various rooms in a building, such as a home. , an office or similar other spaces such as retail. In embodiments, the proximately located controller may be an intelligent LED bulb disposed in the ceiling of a room as depicted in FIG. 5, (or a home server) based on sensor data provided by multiple ones of these small size sensor hubs 100. As described earlier, intelligent light bulb is the subject matter of co-pending U.S. non-provisional application <attorney docket 127075-205200>, contemporaneously filed. In embodiments, sensor hub 100 may also be provided with security software to make sure the unit is protected from hacking or malicious attacks.

The short distance communication mechanism 114, such as BLE, ZigBee, Z-wave, can be used to communicate with the external controller. The communication link may also be used to download patches, download a new analytics algorithm, update profiles, update the lower and upper control limits, and so forth. The downloads and updates may be initiated from a smartphone, a computer tablet, a laptop computer, a server or a desktop PC (not shown).

Additionally, sensor hub 100 may be configured to use a battery which allows the unit to be operational for about 24 hours even during blackouts. The battery can be replaceable, rechargeable using 110V AC-DC converter, or charged using induction, or using a super capacitor.

Referring now to FIG. 4, wherein the sensor hub of the present disclosure, in accordance with various alternate embodiments, is illustrated. As illustrated, sensor hub 400, similar to sensor hub 100 may include processor 402, sensors 404-412, communication mechanism 414, and battery 416. Unlike sensor hub 100, sensor hub 400 has a body in the form a face plate 420 where processor 402 and communication mechanism 414 are disposed behind the face plate 420, and sensors 404-412 and battery 416 are integrated with, and exposed through the front surface of the face plate 420. Some face plate embodiments may include wiring (not shown) that contacts the power line to derive power. Other face plate embodiments may obtain power through induction thereby eliminating a direct contact with the power line.

Referring now to FIG. 6, wherein a block diagram of an example architecture of the sensor hub of the present disclosure, in accordance with various embodiments, is illustrated. As shown (and described earlier), sensor hub 600 may include one or more processors or processor cores 602, and persistent memory 604. In embodiments, multiples processor cores 602 may be disposed on one die. For the purpose of this application, including the claims, the terms “processor” and “processor cores” may be considered synonymous, unless the context clearly requires otherwise. Additionally, sensor hub 600 may include communication interfaces 610, such as BLE, Z-wave, ZigBee and so forth, and sensors 608 (smoke, CO, LPG etc.). The elements may be coupled to each other via system bus 606, which may represent one or more buses. In the case of multiple buses, they may be bridged by one or more bus bridges (not shown).

Each of these elements may perform its conventional functions known in the art. In particular, persistent memory 604 may be employed to store a copy of sensor hub logic 622 implementing the operations described earlier, e.g., but not limited to, reading the sensor data, comparing the sensor data to upper/lower limits or using some kind of an algorithm for comparison, transmitting the sensor data and so forth. Sensor hub logic 622 may be implemented in assembler instructions supported by processor(s) 602 or high-level languages, such as, for example, C or a scripting language, that can be compiled into such instructions. The programming instructions may be placed into persistent memory 604 in the factory, or in the field, through, for example, a distribution medium (not shown), such as a compact disc (CD), or through communication interface 610 (from a distribution server (not shown)). The number, capability and/or capacity of these elements 602-610 may vary from embodiments to embodiments. The constitutions of these elements 602-610 are otherwise known, and accordingly will not be further described.

FIG. 7 illustrates an example non-transitory computer-readable storage medium having instructions configured to practice all or selected ones of the operations associated with sensor hub 100, 400 or 600, and so forth, earlier described, in accordance with various embodiments. As illustrated, non-transitory computer-readable storage medium 702 may include a number of programming instructions 704. Programming instructions 704 may be configured to enable a device, e.g., sensor hub 100, 400 or 600, in response to execution of the programming instructions, to perform, e.g., various sensor hub operations earlier described. In alternate embodiments, programming instructions 704 may be disposed on multiple non-transitory computer-readable storage media 702 instead. In still other embodiments, programming instructions 704 may be encoded in transitory computer readable signals. The programming instruction may also include piece of software that protects or encrypts the data in the memory, storage, data being processed, and in communication channel being exposed to the hackers.

Referring back to FIG. 6, for one embodiment, at least one of processors 602 may be packaged together with a computer-readable storage medium having sensor hub logic 622 (in lieu of storing in system memory 604) configured to practice all or selected aspects of sensor hub operations. For one embodiment, at least one of processors 602 may be packaged together with a computer-readable storage medium having sensor hub logic 622 to form a System in Package (SiP). For one embodiment, at least one of processors 602 may be integrated on the same die with a computer-readable storage medium having sensor hub logic 622. For one embodiment, at least one of processors 602 may be packaged together with a computer-readable storage medium having sensor hub logic 622 to form a System on Chip (SoC).

Example 1 may be a sensor hub, comprising a plurality of sensors of a plurality of sensor types; one or more communication interfaces; a processor coupled to the sensors and the one or more communication interfaces to receive and process sensor data from the sensors, and to transmit the sensor data or results of the processing of the sensor data to an external recipient; and a body encasing the sensors, the one or more communication interfaces and the processor, wherein the body includes features to facilitate the sensor hub to be disposed at an electrical outlet.

Example 2 may be example 1, wherein the body may include a plurality of prongs to plug the sensor hub into the electrical outlet and to draw power from the electrical outlet; and the sensor hub further includes a transformer to step down voltage of the electrical outlet for the sensors, one or more communication interfaces or the processor.

Example 3 may be example 1, wherein the body may have a round form factor with dimensions of a U.S. dollar coin.

Example 4 may be example 1, wherein the body may have a form factor of an electrical outlet face plate.

Example 5 may be example 4, where the sensor hub may further comprise electrical wires to electrically couple the sensor hub to the electrical outlet to draw power from the electrical outlet; and the sensor hub may further include a transformer to step down voltage of the electrical outlet for the sensor, one or more communication interfaces or the processor.

Example 6 may be example 4, wherein the sensor hub may further comprise an inductor to inductively draw power from the electrical outlet.

Example 7 may be example 1, wherein the sensors may comprise a smoke sensor, a CO sensor, a LPG sensor, a motion sensor, a temperature sensor and a humidity sensor.

Example 8 may be example 7, wherein the sensors may further comprise a Radon sensor, a Methane sensor or an Ozone sensor.

Example 9 may be example 1, wherein the one or more communication interfaces may be selected from a group consisting of Bluetooth Low Energy, Z-wave and ZigBee communication interfaces.

Example 10 may be example 1, wherein the external recipient may be an intelligent LED Bulb.

Example 11 may be example 1, wherein the external recipient may be a mobile phone, a computing tablet, a laptop computer, a desktop computer, a set-top box, a game console or a server.

Example 12 may be example 1, further comprising a rechargeable battery cell to provide backup power for the sensor, one or more communication interfaces or the processor.

Example 13 may be example 1, further comprising sensor hub logic to be operated by the processor to collect the sensor data from the sensors, to provide the sensor data to the external recipient, or to compare the sensor data to a plurality of thresholds and report results of the comparison to the external recipient.

Example 14 may be any one of examples 1-13, further comprising security software to be operated by the processor to protect the sensor hub from malicious attacks.

Example 15 may be a method for collecting environmental data, comprising: sensing, with a hub of sensors of a plurality of sensor types disposed at an electrical outlet, a plurality of environment data; processing or analyzing, with the hub of sensors, the plurality of environmental data; and selectively transmitting, by the sensor hub, the environment data or results of the processing or analyzing, to an external recipient.

Example 16 may be example 15, further comprising plugging the sensor hub into the electrical outlet with a plurality of prongs disposed on a body of the sensor hub to draw power from the electrical outlet; and stepping down voltage of the electrical outlet, with a transformer of the sensor hub, for sensors, one or more communication interfaces or a processor of the sensor hub.

Example 17 may be example 15, wherein the sensor hub may have a form factor of an electrical outlet face plate, and the method may further comprise electrically coupling wiring of the sensor hub to the electrical outlet to draw power from the electrical outlet; and stepping down voltage of the electrical outlet, with a transformer of the sensor hub, for sensors, one or more communication interfaces or a processor of the sensor hub.

Example 18 may be example 15, further comprising inductively drawing, with an inductor of the sensor hub, power from the electrical outlet.

Example 19 may be example 15, wherein sensing may comprise sensing with a smoke sensor, a CO sensor, a LPG sensor, a motion sensor, a temperature sensor, a humidity sensor, and at least a selected one of a Radon sensor, a Methane sensor or an Ozone sensor.

Example 20 may be example 15, wherein transmitting may comprise transmitting in accordance with one or more communication protocols selected from a group consisting of Bluetooth Low Energy, Z-wave and ZigBee communication protocols.

Example 21 may be example 15, wherein transmitting may comprise transmitting the environment data or results of the processing or analyzing to an intelligent LED Bulb.

Example 22 may be example 15, wherein transmitting may comprise transmitting the environment data or results of the processing or analyzing to a mobile phone, a computing tablet, a laptop computer, a desktop computer, a set-top box, a game console or a server.

Example 23 may be any one of examples 15-22, wherein processing or analyzing may comprise comparing the environmental data to a plurality of thresholds and transmitting comprises reporting results of the comparison to the external recipient.

Example 24 may be at least one computer-readable storage medium comprising a plurality of instructions configured to cause a sensor hub disposed at an electrical outlet, in response to execution of the instructions by the sensor hub, to read from a plurality of sensors of a plurality of sensor types integrated with the sensor hub, a plurality of environment data; process or analyze the plurality of environmental data; and selectively transmit the environmental data read or results of the process or analysis to an external recipient.

Example 25 may be example 24, wherein to sense may comprise to sense with a smoke sensor, a CO sensor, a LPG sensor, a motion sensor, a temperature sensor, a humidity sensor, and at least a selected one of a Radon sensor, a Methane sensor or an Ozone sensor.

Example 26 may be example 24, wherein to transmit may comprise to transmit in accordance with one or more communication protocols selected from a group consisting of Bluetooth Low Energy, Z-wave and ZigBee communication protocols.

Example 27 may be example 24, wherein to transmit may comprise to transmit the environment data or results of the processing or analyzing to an intelligent LED Bulb.

Example 28 may be example 24, wherein to transmit may comprise to transmit the environment data or results of the processing or analyzing to a mobile phone, a computing tablet, a laptop computer, a desktop computer, a set-top box, a game console or a server.

Example 29 may be example 24-28, wherein to process or analyze may comprise to compare the environmental data to a plurality of thresholds and to transmit comprises to report results of the comparison to the external recipient.

Example 30 may be an apparatus for collecting environmental data, comprising: sensor means to be disposed at an electrical outlet for sensing a plurality of environment data; means for processing or analyzing the plurality of environmental data; and means for selectively transmitting the environment data or results of the processing or analyzing, to an external recipient.

Example 31 may be example 30, wherein means for sensing may comprise means for plugging the sensor means into the electrical outlet to draw power from the electrical outlet; and means for stepping down voltage of the electrical outlet, for sensors, one or more communication interfaces or a processor of the sensor means.

Example 32 may be example 30, wherein means for sensing may comprise means for electrically coupling the sensor hub to the electrical outlet to draw power from the electrical outlet; and means for stepping down voltage of the electrical outlet for sensors, one or more communication interfaces or a processor of the sensor hub.

Example 33 may be example 30, further comprising means for inductively drawing power from the electrical outlet.

Example 34 may be example 30, wherein means for sensing may comprise means for sensing smoke, means for sensing CO, means for sensing LPG, means for sensing motion, means for sensing temperature, means for sensing humidity, and at least a selected one of means for sensing Radon, means for sensing Methane sensor or means for sensing Ozone.

Example 35 may be example 30, wherein means for transmitting may comprise means for transmitting in accordance with one or more communication protocols selected from a group consisting of Bluetooth Low Energy, Z-wave and ZigBee communication protocols.

Example 36 may be example 30, wherein means for transmitting may comprise means for transmitting the environment data or results of the processing or analyzing to an intelligent LED Bulb.

Example 37 may be example 30, wherein means for transmitting may comprise means for transmitting the environment data or results of the processing or analyzing to a mobile phone, a computing tablet, a laptop computer, a desktop computer, a set-top box, a game console or a server.

Example 38 may be any one of examples 30-37, wherein means for processing or analyzing may comprise means for comparing the environmental data to a plurality of thresholds and means for transmitting comprises means for reporting results of the comparison to the external recipient.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims.

Where the disclosure recites “a” or “a first” element or the equivalent thereof, such disclosure includes one or more such elements, neither requiring nor excluding two or more such elements. Further, ordinal indicators (e.g., first, second or third) for identified elements are used to distinguish between the elements, and do not indicate or imply a required or limited number of such elements, nor do they indicate a particular position or order of such elements unless otherwise specifically stated.

What is claimed is:

1. A sensor hub, comprising:
a plurality of sensors of a plurality of sensor types; one or more communication interfaces;
a processor coupled to the sensors and the one or more communication interfaces to receive and process sensor data from the sensors, and to transmit the sensor data or results of the processing of the sensor data to an external recipient; and
a body encasing the sensors, the one or more communication interfaces and the processor, wherein the body includes features to facilitate the sensor hub to be disposed at an electrical outlet.

2. The sensor hub of claim 1, wherein the body includes a plurality of prongs to plug the sensor hub into the electrical outlet and to draw power from the electrical outlet; and the sensor hub further includes a transformer to step down voltage of the electrical outlet for the sensors, one or more communication interfaces or the processor.

3. The sensor hub of claim 1, wherein the body has a round form factor with dimensions of a U.S. dollar coin.

4. The sensor hub of claim 1, wherein the body has a form factor of an electrical outlet face plate.

5. The sensor hub of claim 4, where the sensor hub further comprises electrical wires to electrically couple the sensor hub to the electrical outlet to draw power from the electrical outlet; and the sensor hub further includes a transformer to step down voltage of the electrical outlet for the sensor, one or more communication interfaces or the processor.

6. The sensor hub of claim 4, wherein the sensor hub further comprises an inductor to inductively draw power from the electrical outlet.

7. The sensor hub of claim 1, wherein the sensors comprise a smoke sensor, a CO sensor, a LPG sensor, a motion sensor, a temperature sensor and a humidity sensor.

8. The sensor hub of claim 7, wherein the sensors further comprises a Radon sensor, a Methane sensor or an Ozone sensor.

9. The sensor hub of claim 1, wherein the one or more communication interfaces are selected from a group consisting of Bluetooth Low Energy, Z-wave and ZigBee communication interfaces.

10. The sensor hub of claim 1, wherein the external recipient is an intelligent LED Bulb.

11. The sensor hub of claim 1, wherein the external recipient is a mobile phone, a computing tablet, a laptop computer, a desktop computer, a set-top box, a game console or a server.

12. The sensor hub of claim 1, further comprising a rechargeable battery cell to provide backup power for the sensor, one or more communication interfaces or the processor.

13. The sensor hub of claim 1, further comprising sensor hub logic to be operated by the processor to collect the sensor data from the sensors, to provide the sensor data to the external recipient, or to compare the sensor data to a plurality of thresholds and report results of the comparison to the external recipient.

14. The sensor hub of claim 1, further comprising security software to be operated by the processor to protect the sensor hub from malicious attacks.

15. A method for collecting environmental data, comprising:
sensing, with a hub of sensors of a plurality of sensor types disposed at an electrical outlet, a plurality of environment data;
processing or analyzing, with the hub of sensors, the plurality of environmental data; and
selectively transmitting, by the sensor hub, the environment data or results of the processing or analyzing, to an external recipient.

16. The method of claim 15, further comprising plugging the sensor hub into the electrical outlet with a plurality of prongs disposed on a body of the sensor hub to draw power from the electrical outlet; and stepping down voltage of the electrical outlet, with a transformer of the sensor hub, for sensors, one or more communication interfaces or a processor of the sensor hub.

17. The method of claim 15, wherein the sensor hub has a form factor of an electrical outlet face plate, and the method further comprises electrically coupling wiring of the sensor hub to the electrical outlet to draw power from the electrical outlet; and stepping down voltage of the electrical outlet, with a transformer of the sensor hub, for sensors, one or more communication interfaces or a processor of the sensor hub.

18. The method of claim 15, further comprising inductively drawing, with an inductor of the sensor hub, power from the electrical outlet.

19. The method of claim 15, wherein sensing comprises sensing with a smoke sensor, a CO sensor, a LPG sensor, a motion sensor, a temperature sensor, a humidity sensor, and at least a selected one of a Radon sensor, a Methane sensor or an Ozone sensor; and wherein transmitting comprises transmitting in accordance with one or more communication protocols selected from a group consisting of Bluetooth Low Energy, Z-wave and ZigBee communication protocols.

20. The method of claim 15, wherein processing or analyzing comprises comparing the environmental data to a plurality of thresholds and transmitting comprises reporting results of the comparison to an intelligent LED Bulb, a mobile phone, a computing tablet, a laptop computer, a desktop computer, a set-top box, a game console or a server.

21. At least one computer-readable storage medium comprising a plurality of instructions configured to cause a sensor hub disposed at an electrical outlet, in response to execution of the instructions by the sensor hub, to
read from a plurality of sensors of a plurality of sensor types integrated with the sensor hub, a plurality of environment data;
process or analyze the plurality of environmental data; and
selectively transmit the environmental data read or results of the process or analysis to an external recipient.

22. The at least one computer-readable storage medium of claim 21, wherein to sense comprises to sense with a smoke sensor, a CO sensor, a LPG sensor, a motion sensor, a temperature sensor, a humidity sensor, and at least a selected one of a Radon sensor, a Methane sensor or an Ozone sensor; and wherein to transmit comprises to transmit in accordance with one or more communication protocols selected from a group consisting of Bluetooth Low Energy, Z-wave and ZigBee communication protocols.

23. The at least one computer-readable storage medium of claim 21, wherein to transmit comprises to transmit the environment data or results of the processing or analyzing to an intelligent LED Bulb.

24. The at least one computer-readable storage medium of claim 21, wherein to transmit comprises to transmit the environment data or results of the processing or analyzing to a mobile phone, a computing tablet, a laptop computer, a desktop computer, a set-top box, a game console or a server.

25. The at least one computer-readable storage medium of claim 21, wherein to process or analyze comprises to compare the environmental data to a plurality of thresholds and to transmit comprises to report results of the comparison to the external recipient.
